(19) United States
(12) Patent Application Publication
(10) Pub. No.: US 2023/0414315 A1
(43) Pub. Date: Dec. 28, 2023

(54) DISPOSITIF DE NETTOYAGE POUR INSTRUMENTS DENTAIRES, MÉDICAUX OU CHIRURGICAUX

(71) Applicant: PRODUITS DENTAIRES SA, Vevey (CH)

(72) Inventors: Shanon PATEL, Vevey (CH); Mel OLLIERIC, Vevey (CH); Nicolas GEHRIG, Vevey (CH)

(21) Appl. No.: 18/339,671

(22) Filed: Jun. 22, 2023

(30) Foreign Application Priority Data
Jun. 23, 2022 (WO) PCT/IB2022/055829

(51) Int. Cl. A61B 90/70; B08B 1/00; B08B 3/08; B08B 3/00; A61L 2/26; A61L 2/18; A61L 2/22

(52) U.S. Cl. CPC A61B 90/70; B08B 1/005; B08B 3/08; B08B 3/003; A61L 2/26; A61L 2/18; A61L 2/22; A61L 2202/17; A61L 2202/122; A61L 2202/121; A61L 2202/24

(57) ABSTRACT

Disclosed is a device for cleaning dental, medical or surgical instruments including a support, a receptacle mounted on the support and intended to receive at least the active part of a dental, medical or surgical instrument and a cover arranged to be removably mounted on one end of the receptacle. The cover includes at least one slot, the slot being arranged such that at least the active part of the instrument can be inserted into the receptacle and removed from the receptacle through the slot, the friction between the active part of the instrument and at least one wall of the slot when the instrument passes through the slot allowing the active part to be mechanically cleaned by retaining solid or liquid dirt and deposits.

### CROSS-REFERENCE TO RELATED APPLICATIONS

This application claims the priority under 35 USC 119(a) of international patent application PCT/IB2022/055829 filed on Jun. 23, 2022, the entirety of which is incorporated herein by reference.

### BACKGROUND OF THE INVENTION

### Field of the Invention

The present invention relates to a device for cleaning dental, medical or surgical instruments.

### Description of the Related Art

During dental, endodontic or periodontic treatment, more or less viscous liquids or deposits accumulate along the instruments used (bur, file, scraper or scaler). One option for cleaning the instruments is to provide a cloth arranged on a plate on which the practitioner wipes the active part of his/her instrument to clean it. In order to increase comfort for the practitioner, devices have been proposed which are to be placed on a finger or hand of the practitioner opposite the hand holding the instrument. The device can, in particular, take the form of a ring having a cleaning zone on which it is possible to rub the active part of the instrument. Such a device is for example described in US 2008/0311543. This cleaning method which consists of rubbing the active part of the instrument on a cleaning surface is particularly suitable for scraper- or scaler-type instruments.

There are also ring-shaped devices that the practitioner can slip onto one of his/her fingers and which comprise a support intended to receive a foam pad into which the instruments provided for the dental treatment can be jabbed. Therefore, the instruments are immediately accessible by the practitioner and, by removing an instrument from the foam pad, the deposits which were attached to said instrument remain trapped in the foam which enables the instrument to be cleaned. To perfect the cleaning, it is also possible to imbibe the foam pad with a disinfectant. Document US2011229843 describes, for example, such a device.

These devices thus offer a solution for cleaning instruments, in particular endodontic instruments, but also a solution for accessing and storing the instruments during treatment and in particular dental treatment. In fact, during dental and endodontic treatment in particular, the practitioner needs several instruments for accessing, shaping, cleaning and enlarging the root canal. These instruments are used sequentially according to their type and size. The sequence of instruments is determined based on the case to be treated and can be prepared in advance by the practitioner or another person. Easy access to these instruments is important, to allow in particular the practitioner to change instruments quickly and comfortably during the treatment. The ring-shaped devices described above having a foam pad into which the instruments are jabbed are thus used to store the instruments and facilitate the preparation of the instruments and access thereto during treatment. This solution is in particular more comfortable and ergonomic than simply arranging the instruments on a plate.

However, the use of a foam pad into which the instruments are jabbed is not without problems: on the one hand, it is not easy—in such a pad—to distinguish between the precise locations for the instruments, which runs the risk of losing the order of the instruments for the treatment or mixing up clean and dirty instruments during the treatment. Furthermore, the endodontic instruments are generally tapered and very thin at their active part and there is a risk of snapping or bending said active part of the instrument when jabbing it into the foam. This is because this foam must be sufficiently rigid to hold the instrument and aid in the mechanical cleaning thereof when the instrument is removed from the pad. Moreover, by plunging the active part of a clean instrument into the foam pad, the deposits are likely to move back up the instrument, which is not desirable.

The problem of cleaning and/or arranging the instruments also arises during periodontic treatments (including scaling) or other dental, medical or surgical treatments during which instruments must be regularly cleaned and/or stored within reach of the user.

### SUMMARY OF THE INVENTION

The aim of the present invention is to provide a device for cleaning dental, medical or surgical instruments which is efficient, ergonomic, easy to use and easy to manufacture. Another aim of the present invention is to provide a device which also allows easy, efficient and ergonomic arranging and storing of the instruments, the device being able to be used as a support for at least one but preferably a sequence of instruments which are necessary during dental, medical or surgical treatment.

The present invention relates to a device for cleaning dental, medical or surgical instruments as disclosed and claimed.

### BRIEF DESCRIPTION OF THE DRAWINGS

The appended drawings illustrate schematically and by way of example several embodiments of the invention.

FIGS. 1 to 3 illustrate a device for cleaning dental, medical or surgical instruments according to a first embodiment of the invention, seen from the side, from the top and from the bottom respectively.

FIG. 4 illustrates a part of the device of FIG. 1, seen from the side.

FIGS. 5, 6 and 7a and 7b illustrate the cover of the device according to the preceding figures, seen from the top, from the bottom, from the side and as a cross-section respectively.

FIG. 8 illustrates a variant of the device of FIG. 3, seen from the bottom.

FIG. 9a is similar to FIG. 1 whilst FIG. 9b illustrates the device of FIG. 9a in an intermediate position in which said device can be easily positioned on a finger by a user.

FIGS. 10 to 12 illustrate a second embodiment of the device for cleaning dental, medical or surgical instruments according to the invention, seen from the side, from the top and as a cross-section respectively.

FIGS. 13 to 17 illustrate a third embodiment of the device for cleaning dental, medical or surgical instruments according to the invention.

FIGS. 13 and 14 illustrate the device as a perspective view and seen from the side.

FIG. 15 is a cross-sectional view of FIG. 14.

FIGS. 16 and 17 are exploded views, FIG. 17 being a cross-sectional view.

### DETAILED DESCRIPTION

The device in accordance with the invention is in particular intended to clean dental, medical or surgical instruments intended in particular to scrape, grind, cut, debride, excise, clean . . . Such an instrument generally comprises a first portion intended to be gripped by a user or to be inserted into another tool (handle, contra-angle attachment, motor . . . ). This first portion is generally not active, i.e. it does not contribute to the work for which the instrument is designed. The instrument further comprises an active part extending as a continuation of the first portion and having means making it possible to perform the work or treatment for which the instrument is designed. For example, this active part can have cutting edges, a hook, a gouge, a small grinding ball . . . In the case of an endodontic instrument, this active part—which may or may not be tapered—has a relatively small diameter.

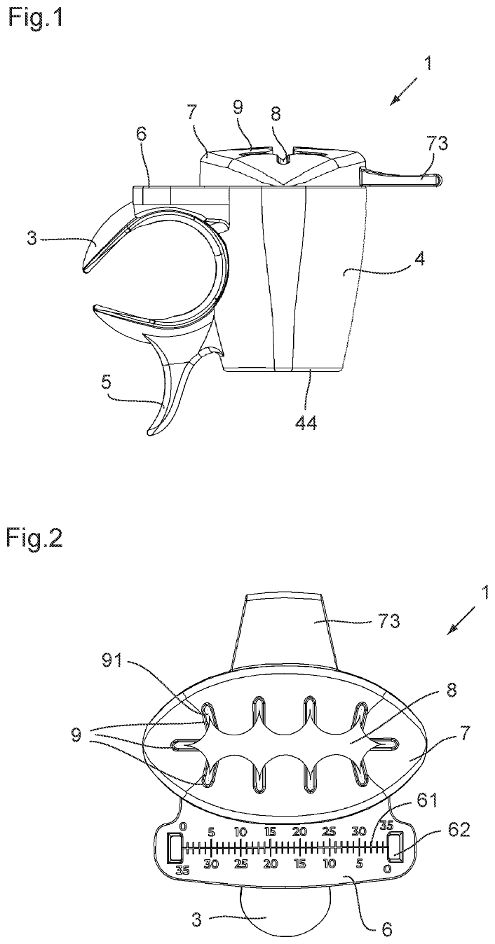

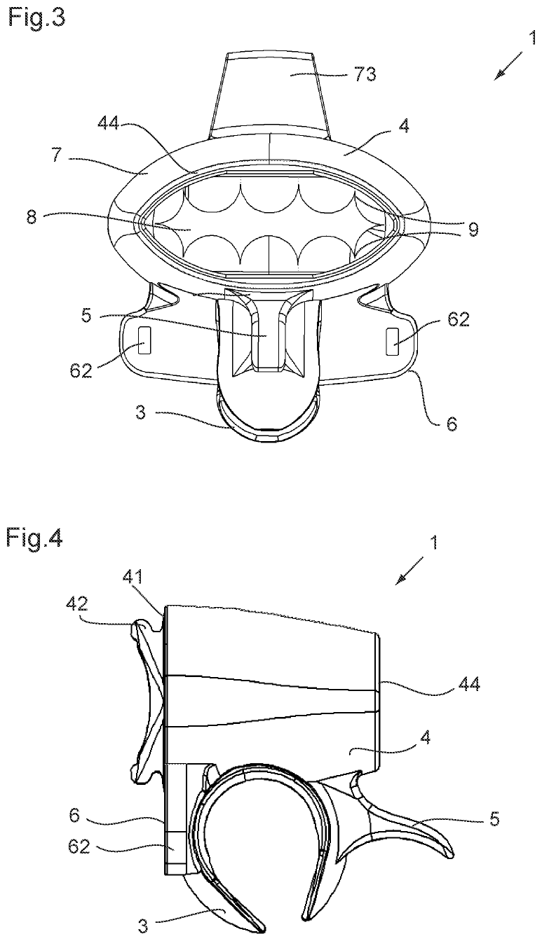

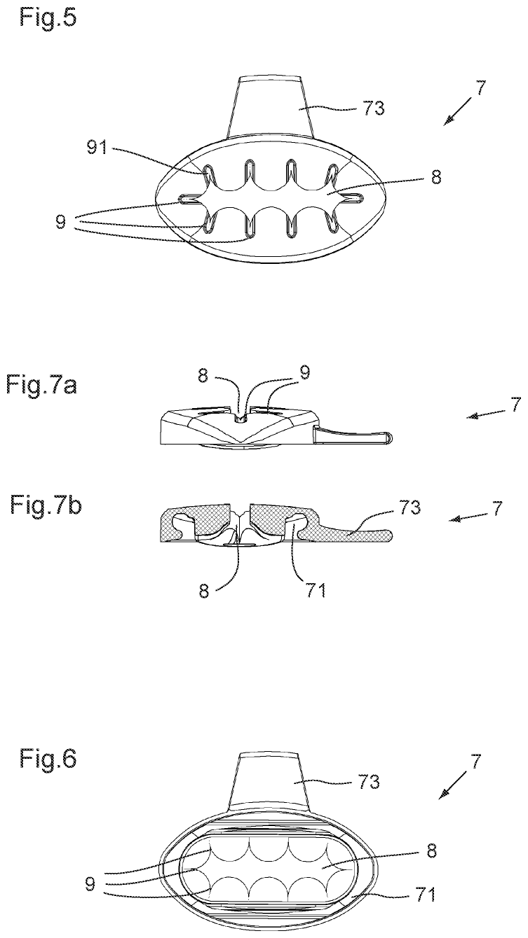

In the first embodiment of the invention as illustrated in FIGS. 1 to 9b, the cleaning device 1 in accordance with the invention is intended to be used in dentistry for endodontic and periodontic instruments.

The device 1 is intended to be positioned on a finger of a hand of a user during dental treatment and in particular endodontic treatment. The device 1 is intended to receive one or more dental instruments for the arrangement and cleaning thereof during the treatment.

Generally, the cleaning device in accordance with the invention comprises a support on which a receptacle is mounted. In the first embodiment, the device 1 is intended to be slipped onto the finger of a user and the support thus has the shape of a ring 3 through which a finger passes. As illustrated, the ring 3 is open but, as a variant, this ring could be closed. Preferably, the ring 3 further comprises an extension 5 arranged to prevent the device 1 from pivoting once said device 1 is placed on the finger of a user.

In the first embodiment illustrated in FIGS. 1 to 7b, the receptacle 4 is open at both of its ends. Its shape and in particular its length are such that it is able to receive the dental instrument, and in particular its entire active part, and preferably at least one part of the first portion of the instrument.

According to the invention, the device 1 further comprises a cover 7 arranged to be removably mounted on the receptacle 4 and intended to at least partially close a first end 41 of the receptacle 4. Preferably, the cover 7 is flexible and elastic, in the manner of a membrane. At its first end 41, the receptacle 4 comprises protrusions 42 intended to cooperate with corresponding grooves 71 on the cover 7. Preferably, the cover 7 further comprises a tab 73 arranged to facilitate the mounting and removing of the cover 7 on/from the receptacle 4 and in particular the engagement of the protrusions 42 in the grooves 71 by deforming/stretching the cover 7.

The cover 7 has a central opening 8, the size of which is large enough to allow the passage of the dental instrument without the instrument touching the walls of the opening 8. By way of example, the conventional dental instruments have an active part, the diameter of which varies between a few hundredths of a millimeter and less than 3 millimetres. The cover 7 further has at least one slot 9 communicating with the opening 8. As illustrated, the cover 7 comprises ten slots 9 which are all open at one of their ends to the central opening 8. Each slot 9 is sized such that it is necessary to exert a force on the dental instrument to make it pass through the slot 9 and such that its active part rubs against the walls of the slot through which it passes. Therefore, by passing the dental instrument through the slot in a manner substantially perpendicular thereto, it is possible to mechanically clean the active part, the more or less viscous liquids or deposits present on said active part being retained by the slot 9.

Preferably, the slots 9 are also sized to make it possible to retain the dental instrument and in particular its first portion and thus to act as a housing for the dental instruments for the arranging thereof during dental treatment.

Preferably, the cover 7 is obtained by injection moulding in a material which allows it to be relatively flexible, such as TPE or silicone. In the embodiment illustrated in the figures, the cover 7 has a greater thickness on its periphery at the locations of the grooves 71, whilst the thickness of the central portion in which the central opening 8 and the slots 9 are located is thinner. Moreover, the slots 9 have rims 91, the thickness of which is thinner than the cover 7 such that these rims 91 form flexible blades allowing the cleaning of the active part of a dental instrument to be further improved, in the manner of a glass wiper.

Preferably, the device 1 further comprises a ruler 6 arranged on the ring 3 to make it possible to measure the active length of a dental instrument or even the length of a gutta-percha cone. Preferably, the ruler 6 comprises a millimeter gradation 61 arranged to be able to be used from right to left or from left to right depending upon the hand or side of the user where the device 1 is located. Preferably, the ruler 6 comprises at each end of the gradation 61, a hole 62 in which a portion of a stopper element can be housed, said stopper element intended to be slid along the dental instrument to delimit the length of the working portion of the active part of the instrument. Owing to the hole 62 and the gradation 61, it is thus possible to position a stopper element precisely along the instrument and in particular its active part:
the stopper element is first slipped onto the instrument (either by the user at the time of treatment or by the manufacturer of the instrument), in particular onto its active part;
it is then positioned in one of the holes 62 (depending upon the hand or side where the device 1 is located);
with the stopper element locked in the hole 62, it is possible to slide the instrument through said stopper element facing the gradation 61 until said stopper element is positioned at the correct length on the active part of the instrument.

Preferably, and as illustrated in the figures, the ring 3, the receptacle 4 and the ruler 6 are made from one piece which can be made from any suitable material, but preferably synthetic material. As a variant, these pieces could be assembled together to form the device 1 and be made from different materials.

In order to use the device 1, the user places the cover 7 onto the end 41 of the receptacle 4. In the illustrated embodiment, this step is facilitated by the tab 73, the arrangement of the protrusions 42 and grooves 71, and by the fact that the cover 7 is flexible. It is in fact possible to position some of the grooves 71 in the protrusions 42 and then finish placing the cover 7 by deforming it by pulling on the tab 73 so that all the protrusions 42 take their place in the corresponding grooves 71. The cover 7 is thus mounted on the receptacle 4.

At this stage, it is already possible to position the dental instruments provided for a particular treatment on the device 1. To do this, the user handles the instrument via its first portion and inserts it into the receptacle 4 through the central opening 8 of the cover 7 in a direction parallel to the longitudinal axis of the receptacle 4. Once all of the active part of the instrument is located in the receptacle 4, the instrument is then moved/slid into a slot 9 of the cover 7 in a direction parallel to the longitudinal axis of said slot 9 to be retained in this slot 9. Preferably, it is the first portion of the instrument—which is not active—which is retained in the slot 9. As will be seen hereinafter, this makes it possible to ensure complete cleaning of all of the active part. Therefore, owing to the central opening 8 of the cover 7, there is no force exerted onto the very fragile tip of the instrument when it is inserted into the receptacle 4 of the device 1. The slots 9 for their part can be used as a support for the instruments for the comfortable and efficient arranging thereof during the treatment. Moreover, the slots 9 offer well-defined positions which make it possible to clearly identify the place of an instrument in a sequence or the condition of an instrument (used or not used, clean or dirty for example).

Then, the user can position the device 1 on one of his/her fingers, generally the index finger of the left hand for right-handed users and the index finger of the right hand for left-handed users, by sliding the chosen finger into the ring 3.

In the in-use position of the device 1 on a finger of the user, the extension 5 comes to bear against the finger adjacent to the finger onto which the ring 3 is slipped, and thus prevents the device 1 from turning. Preferably, and as illustrated in FIGS. 9a and 9b, the extension 5 and the ring 3 also allow easier placement of the device 1 on the finger of the user. In fact, the ring 3 and the extension 5 are preferably shaped such that the user can exert a force on the extension 5 so as to deform the ring 3 to enlarge it, as illustrated in FIG. 9b. It is thus easier for the user to position the device 1 and slip the ring 3 onto the chosen finger. This deformation of the ring 3 is shown in FIGS. 9a (rest position) and 9b (deformed position).

Once the device is positioned on the finger of the user, said device 1 is ready to be used. Either the device 1 is already equipped with the instrument(s) necessary for the proposed dental treatment, or the user can also position the instruments in the device 1 at that moment once the device 1 is placed in his/her hand, according to the procedure described above.

During the treatment, the user has easy access to the required instruments: the user can remove the suitable instrument simply by pulling it out of the receptacle 4 through the slot 9. Furthermore, the user can also use the ruler 6 for measuring the suitable length of the active part of the instrument and/or place or set the position of a stopper element on said active part using holes 62 to thereby define the appropriate working length of the active part.

In order to clean a dirty instrument, the user will insert the instrument into the receptacle 4 via the central opening 8 of the cover 7. Therefore, given the shape and size of this central opening 8, no force is exerted on the fragile tip of the instrument and no deposits move back up the instrument since said instrument, and in particular the active part thereof, will not touch the edges of the central opening 8. Then, the user moves the instrument in translation in the receptacle 4 into one of the slots 9. Preferably, all of the active part of the instrument is inserted into the receptacle 4 such that it is the first portion of the instrument—which is not active—which comes into contact with the slot 9 during the translation of the instrument. Therefore, damage to the active part and/or the slot is avoided during this translation and the active part of the instrument, the part comprising the deposits, is located entirely within the receptacle 4 beneath a slot 9 of the cover 7. By pulling the instrument through the slot 9 so that the active part leaves the receptacle 4, the walls of the slot 9 rub against said active part and exert their mechanical cleaning action. Of course, it is possible to move the instrument through a slot 9 several times to guarantee a good cleaning effect. The removed liquid or solid deposits remain trapped beneath the cover 7 and in the receptacle 4 and cannot re-contaminate the active part of an instrument during subsequent cleaning steps.

Preferably, the device 1 is designed such that, at the end of the treatment, the main body which is made from one piece and is formed of the ring 3, the extension 5, the receptacle 4 and the ruler 6 can be cleaned and sterilised ready for the next use. In a preferential manner, the main body is designed to withstand several tens of sterilisation processes. The cover 7, for its part, is preferably disposable. As a variant, it could also be designed to be reused after sterilisation.

The device 1 described above is particularly well suited for manual or rotatable endodontic instruments (of which the first portion is intended to be fixed in a contra-angle attachment for the rotation thereof). These instruments, intended to be fixed in an auxiliary handle, thus have a size which is particularly suitable for being stored in the device in accordance with the invention. With regard to the scrapers or other scalers of which the first portion often takes the form of a handle which the user can grab during the treatment, although it is not possible to store these instruments in the device in accordance with the invention owing to the size of the instrument and in particular its length, it is still possible to clean the active part of these instruments by inserting them into the receptacle 4 and passing them through the slots 9 of the cover to thereby mechanically remove the solid or liquid deposits. In this sense, the device in accordance with the invention first of all makes it possible to clean dental, medical or surgical instruments and, depending upon the size of the instruments, also makes it possible to store said instruments.

The device 1 could also comprise any support or accessory which is useful during dental, medical or surgical treatment, such as a container for a gutta-percha cone or for sealing cement or for a disinfectant solution (for example imbibed in a foam). Such a foam imbibed with disinfectant could also be located within the receptacle 4. Said receptacle 4 could be fully closed at its second end 44 opposite the first end 41 so as to hold a disinfectant liquid.

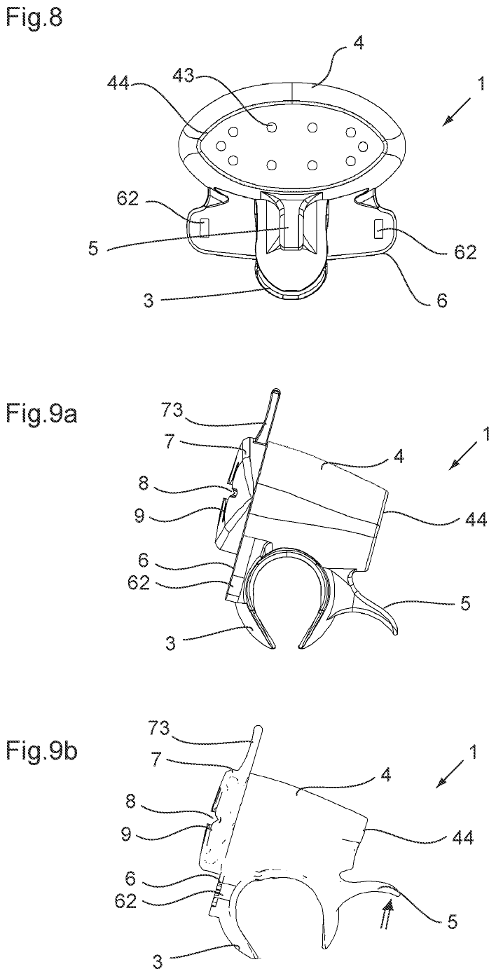

FIG. 8 illustrates a variant of the above embodiment in which the receptacle 4 is partially closed at its second end 44 opposite the first end 41 and has openings 43 facilitating cleaning and sterilisation.

As another variant, the ring 3 could be closed or arranged to have an adjustable size by deformation or by another suitable adjustment means.

Preferably, the device 1 is arranged to be able to be placed on a flat surface such that the instruments housed in the slots 9 of the cover 7 do not touch said surface.

In the embodiment illustrated in the figures, the cover 7 has a central opening 8 communicating with each of the slots 9. As a variant, the cover 7 could have several openings with a larger diameter than the slots 9, each communicating with one or more of said slots 9. For example, each slot 9 could end with a larger opening, through which the active part of an instrument can pass without hitting the walls of said opening.

Whilst the opening(s) of the cover have the advantage of reducing the risks of the active part of the instrument breaking and of limiting the amount that the deposits move back up the instrument during the insertion thereof into the receptacle through the cover, these openings are not essential to the functions of storing and cleaning the instruments. In fact, as a variant, the cover 7 could comprise only one or multiple slots 9. In this case, the instruments are inserted into the receptacle 4 through the slots 9 directly, the cleaning and storing of the instruments in the slot 9 working in the same way as the principle described above.

The device as illustrated in the figures could also be used without the cover 7 to store gutta-percha cones in the receptacle 4 and to measure them using the ruler 6.

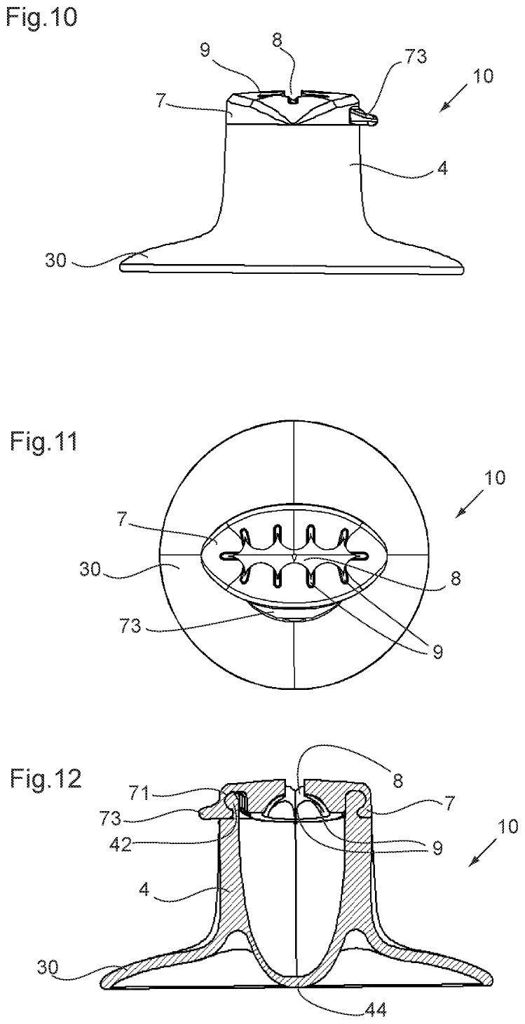

FIGS. 10 to 12 illustrate a second embodiment of the device for cleaning dental, medical or surgical instruments according to the invention. Elements of this embodiment which are identical or quasi-identical to those of the first embodiment have the same reference numerals.

In this embodiment, the cleaning device 10 is intended to be placed on a substantially flat surface such as a plate or operative field. To this end, the support of the device 10—which had the shape of a ring in the first embodiment—has the shape of a base 30 in this second embodiment.

As in the first embodiment, the receptacle 4 is mounted on the base 30 and the entire structure is preferably made from a single piece. As a variant, the device 10 could also comprise a ruler 6 which is similar to that described in the first embodiment and which could either be mounted on the receptacle 4 or the base 30 or be made from a single piece with these last two elements. In the illustrated embodiment, the receptacle 4 is closed at its end 44 opposite that receiving the cover 7.

The features of the cover 7 and the operation of the device 10 are in every way similar to those described with reference to the first embodiment.

Preferably, the base 30 comprises a non-slip portion to ensure good adherence to the surface on which it is placed. As a variant, the base could comprise any suitable means making it possible to ensure this adherence, such as a silicone or TPE portion, one or more suction cups, one or more feet or a particular shape.

All of the variants and advantages described above with reference to the first embodiment are applicable to this second embodiment. Moreover, the operation and use of the device 10 in accordance with the second embodiment are furthermore similar to that described above.

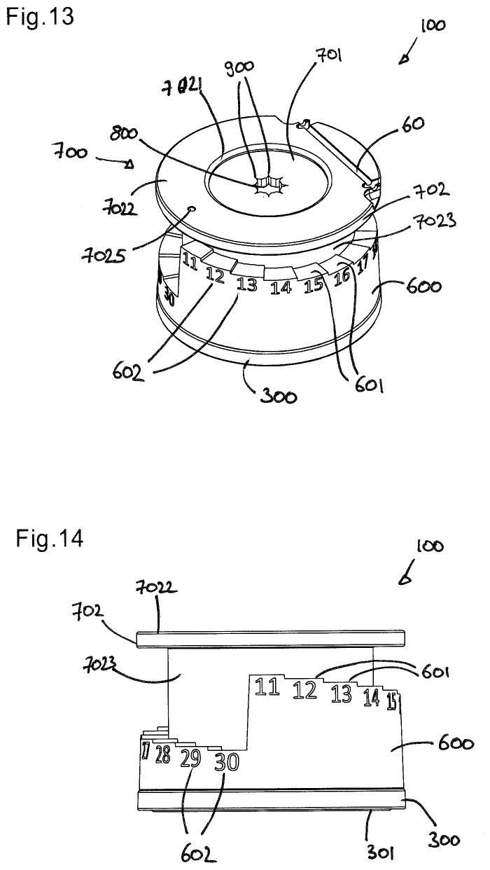

FIGS. 13 to 17 illustrate a third embodiment of the invention. In this embodiment, the cleaning device 100 is intended to be placed on a substantially flat surface such as a plate or operative field. To this end, the support of the device 100—which had the shape of a ring in the first embodiment—has the shape of a base 300 similar to that of the second embodiment.

Preferably, the base 300 comprises a non-slip portion to ensure good adherence to the surface on which it is placed. In the illustrated embodiment, this non-slip portion is formed by an O-ring 301 housed in a groove 302 provided for this purpose on the bottom of the base 300. As a variant, the base could comprise any suitable means making it possible to ensure this adherence, such as a silicone or TPE portion, one or more suction cups, one or more feet or a particular shape.

As in the first embodiment, the receptacle 400 is mounted on the base 300 and the entire structure is preferably made from a single piece. As in the second embodiment, the receptacle 400 is closed at its end 440 opposite that receiving the cover 700 (support-side end 300).

One of the features of this third embodiment resides in the cover 700 being produced in two parts. In fact, the cover 700 according to this third embodiment comprises a first part 701 having the form of a seal, which is preferably flexible and elastic in the manner of a membrane. This first part 701 is intended to be placed on the first end 410 of the receptacle 400 to close it. Preferably, this first part 701 comprises centring means making it possible to ensure the positioning thereof on said first end 410 of the receptacle 400. In the example illustrated in particular in FIG. 14, the first part 701 comprises a groove 7011 on its lower part as well as a rim 7012 at its periphery which are intended to cooperate with the edges of the first end 410 of the receptacle 400 for the positioning and in particularly centring of the first part 701 of the cover 700 when it is placed on said receptacle 400. Any other means for centring or fixing the first part 701 of the cover 700 on the receptacle 400 could be provided, it being understood that these means are totally optional.

The cover 700 further comprises a second part 702 which will be positioned on the receptacle 400 to keep the first part 701 of the cover 700 on the first end 410 of the receptacle 400. As illustrated in FIGS. 13 to 17, the second part 702 has the shape of a ring with a central opening 7021 through which the first part 701 of the cover 700 can be accessed when the device 100 is assembled, a rim 7022 which can for example have a ruler 60 similar to the ruler 6 described with reference to the first embodiment, and an annular portion 7023 intended to surround the receptacle 400 for fixing this second part 702 and thus the cover 700 to said receptacle 400.

As in the previous embodiments, the cover 700 comprises a central opening 800 and slots 900. In particular, in this third embodiment, the central opening 800 and the slots 900 are formed in the first part 701 of the cover 700 and can be accessed through the opening 7021 of the second part 702 of the cover 700 when the device 100 is assembled. The features and operation of these slots and central opening are in every way similar to what was described above with reference to the first embodiment.

Preferably, the first part 701 of the cover 700 is produced from a flexible and elastic material. This first part 701 can in particular be produced from thermoplastic elastomer (TPE), from silicone or in a foam, for example a foam with a sandwich structure. With regard to the second part 702 of the cover 700, this can be produced from stainless steel, from thermoplastic or thermosetting material.

In this embodiment, the first part 701 of the cover can be disposable whereas the second part 702 can preferably be sterilised and reused.

The cover 700 is assembled on the receptacle 400 in the following manner with the receptacle 400 and the support 300 placed on a flat surface (table, tray), the first part 701 of the cover 700 is placed on the first end 410 of the receptacle 400, possibly positioned using the groove 7011 and/or rims 7012. The second part 702 is then inserted onto the receptacle 400 for keeping the first part 701 in position. In general, the second part 702 is pushed onto the receptacle 400 until it abuts against the first end 410 of this receptacle. In order to ensure that the cover 700 is assembled on the receptacle 400, said receptacle 400 comprises a shoulder 401 around which the second part 702 of the cover 700 is forcibly driven. This makes it possible to be able to handle the entirety of the device 100, taking it by said cover 700 (in particular by the second part 702). As a variant, the receptacle and the cover could comprise snap-fit means or any other suitable means enabling the second part of the cover to be assembled on the receptacle.

Once the cover 700 is assembled on the receptacle 400, the slots 900 and/or the opening 800 of the first part 701 of the cover 700 can be accessed through the opening 7021 of the second part 702 of the cover 700. Cleaning a dirty instrument or storing an instrument using the device 100 according to the third embodiment can thus be effected in the same manner as with a device 1 or 10 according to one of the first or second embodiments described above.

All of the variants and advantages described above with reference to the first and second embodiments are applicable to this third embodiment.

Besides the ruler 60 which is located on the second part 702 of the cover 700, FIGS. 13 to 17 also illustrate complementary means for measuring the useful length of a dental instrument which are particularly practical and easy to use. In fact, the device 100 according to the third embodiment further comprises a measuring scale 600 arranged to be rotatable about the receptacle 400 and/or the cover 700 whilst being prevented from being moved in translation with respect to said receptacle 400 when the cover 700 is assembled on the receptacle 400. As illustrated in the figures, this measuring scale 600 has the shape of a staircase and comprises several steps 601 each corresponding to a length to thereby form a gradation. Length indications 602 can be provided on each of the steps or on the side of the measuring scale 600.

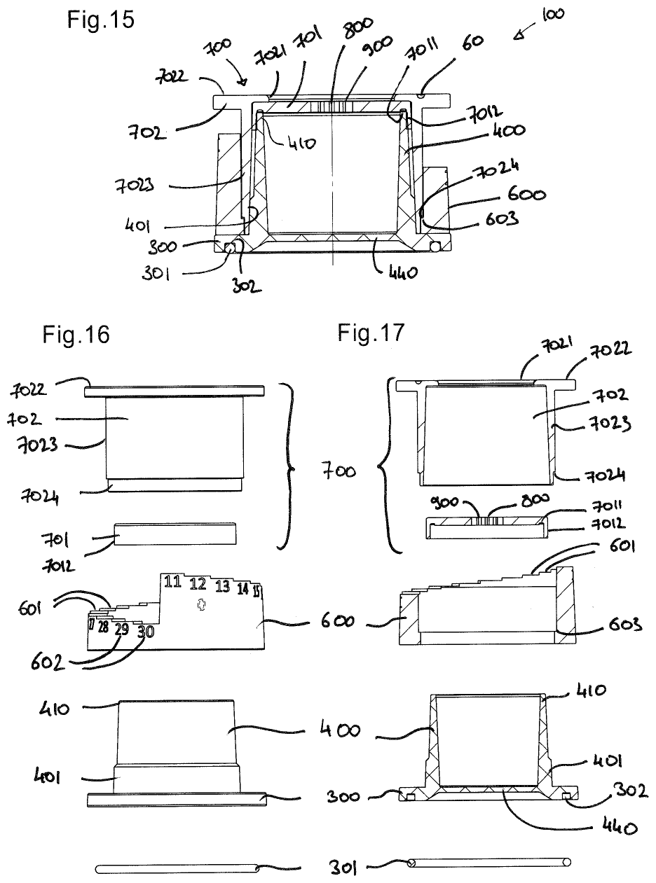

In the illustrated embodiment, the second part 702 of the cover 700 is arranged so that its annular portion 7023 abuts against the support 300 when the cover 700 is mounted on the device 100. This annular portion 7023 comprises, at its end opposite the rim 7022, a first shoulder 7024 arranged to cooperate with a second shoulder 603 on the measuring scale 600 on its lower surface to prevent same from moving in translation on the receptacle 400 (see FIG. 15).

In order to use this measuring scale 600, the cover 700 comprises a hole 7025 on the rim 7022 of its second part 702. The user who wishes to determine the suitable length of the active part of an instrument and in particular to position a stopper element along this active part can thus:
pivot the measuring scale 600 until the step 601 of the desired length is placed beneath the hole 7025 (an indicator can be provided on the annular part 7023 of the second part 702 of the cover 700;
insert said instrument in the hole 7025 and optionally align the axis of the instrument with an axis depicted on the exterior of the annular portion 7023 of the cover 700 to ensure the verticality (this axis can correspond to the indicator mentioned above);
move the stopper element along the active part of the instrument by pushing it until the tip touches the chosen step 601, said stopper element is thus positioned at the desired length on the active part of the instrument.

The measuring means formed by the measuring scale 600 and the hole 7025 can likewise be used to measure the length of a gutta-percha cone and cut said cone to the desired length.

Therefore, these measuring means make it possible to easily adjust the length of the active part of an instrument by limiting the number of operations: a single hole in which the instrument is inserted, followed by the measuring scale which pivots. This also facilitates the step of disinfecting the cleaning device 100.

As a variant, the measuring scale 600 and/or the receptacle 400 and/or the cover 700 can comprise indexing means arranged to determine indexing positions of the measuring scale in which a step 601 of the scale is aligned with the hole 7025 of the cover 700.

Preferably, the measuring scale 600 is produced from stainless steel or from a thermoplastic material (TPE/POM).

Generally, the device in accordance with the invention for cleaning dental, medical or surgical instruments during dental, medical or surgical treatment comprises:
a support;
a receptacle mounted on the support and intended to receive at least one portion of the active part of a dental, medical or surgical instrument; and
a cover arranged to be removably mounted on one end of the receptacle.

The cover comprises at least one slot. Said slot is arranged such that the active part of the instrument can be inserted into the receptacle through the slot and the friction between the active part of the instrument and at least one wall of the slot when said instrument passes through said slot allows said active part to be mechanically cleaned.

Preferably, the slot is also shaped to hold the instrument in the receptacle and to be used as a support therefor when the active part thereof is inserted into the receptacle.

Preferably, the cover further comprises at least one opening communicating with, and being wider than, the slot(s) and through which the instrument and in particular its active part can pass without friction and without constraint.

According to a first variant, the support is a ring which is open or closed or has an adjustable size and which is intended to be slipped onto the finger of a user.

Preferably, the device comprises an extension fixedly attached to the ring and arranged to come to bear against the finger adjacent to the finger passing through the ring when the device is in place so as to prevent the ring and the device from turning.

According to a second variant, the support is a base intended to be placed on a substantially planar surface such as a tray or operative field.

The receptacle can be totally or partially closed at its end opposite that receiving the cover. The receptacle can likewise contain disinfectant means such as a foam imbibed with a disinfectant solution.

The device can further comprise a ruler mounted on the receptacle or the support and having a gradation making it possible to measure the length of the working portion of the active part of an instrument. Preferably, a blind housing or through-going housing (hole) is arranged at the start of said gradation.

Preferably, the cover comprises several slots which can be arranged to form sectors or groups making it possible to classify or sort several instruments according to set criteria (clean and dirty, or even according to the sequence used for the treatment).

Preferably, the cover is flexible and elastic, formed for example from synthetic material, thereby allowing it to be deformed for placement on the receptacle. Moreover, in a preferential manner, it has a tab making it possible to easily mount and remove said cover on/from the receptacle.

Preferably, the support, the receptacle, the ruler and/or the extension are made from a single piece. The piece is made for example from synthetic material or from elastomer and can be sterilised.

Depending on the variants, the cover is made from one or more pieces.

According to some variants, the device further comprises measuring means comprising a hole in a portion of the device in which a tapered rod (instrument or cone) can be inserted without penetrating into the receptacle and a measuring scale arranged to be movable in rotation but not in translation with respect to the receptacle, the measuring scale comprising several steps each determining a particular length.

There is therefore provided a device for cleaning dental, medical or surgical instruments offering a crucial and efficient aide for the cleaning and/or arranging of instruments during dental, medical or surgical treatment. The slot(s) of the cover ensure efficient mechanical cleaning whilst limiting the risks of breakage or deformation of the instruments. This risk is even lower with a wide opening through which the fragile tip of the instruments can pass without friction or constraint. Also in this variant, the cleaning is improved since it is possible to prevent the deposits moving back up the instrument when the instrument is inserted into the receptacle.

The presence of slots on the cover permits a clear arrangement of the instruments and allows the instruments to be distinguished according to set criteria such as level of dirtiness, order of use during the treatment . . . This ordered arrangement is difficult to achieve with a device comprising a foam pad as is known.

The device is also ergonomic, easy to hold, versatile in the sense that it can be used for a large number of treatments and instruments: endodontic, periodontic or other medical treatment, files, bur, scraper, scaler, gutta-percha cone . . . .

The device provides an efficient aid to the user for cleaning and/or arranging instruments during dental, medical or surgical treatment and makes it possible to substantially increase the efficiency of said treatment.

Finally, each element can be made from materials which are easily machinable and by simple and known methods, which makes the device advantageous from a commercial point of view.

1. Device for cleaning dental, medical or surgical instruments comprising an active part, said device comprising:
a support;
a receptacle mounted on the support and intended to receive at least the active part of a dental, medical or surgical instrument; and
a cover arranged to be removably mounted on one end of the receptacle; and wherein the cover comprises at least one slot, said slot being arranged such that at least the active part of the instrument can be inserted into the receptacle and removed from the receptacle through the slot, the friction between the active part of the instrument and at least one wall of the slot when said instrument passes through said slot allowing said active part to be mechanically cleaned by retaining solid or liquid dirt and deposits.

2. The device as claimed in claim 1, wherein the support is a ring intended to receive a finger of a user, the ring being open or closed or having an adjustable size.

3. The device as claimed in claim 1, wherein an extension fixedly attached to the ring is arranged to come to bear against the finger adjacent to the finger passing through the ring when the device is in place so as to prevent the ring and the device from turning in the in-use position of the device.

4. The device as claimed in claim 1, wherein the support is a base intended to be placed on a substantially planar surface.

5. The device as claimed in claim 1, wherein the cover is formed of several pieces.

6. The device as claimed in claim 5, wherein at least one piece of the cover has the form of a seal made from a thin foam, said seal having said at least one slot.

7. The device as claimed in claim 1, wherein the slot is also shaped so as to hold the instrument and be used as a support therefor when the active part is inserted into the receptacle through the slot, a force exerted on the instrument being necessary to cause said instrument to pass through the slot.

8. The device as claimed in claim 1, wherein the cover further comprises at least one opening communicating with, and being wider than, the slots and through which the instrument can pass without friction and without constraint.

9. The device as claimed in any one of the preceding claim 1, wherein the receptacle is at least partially closed at its end opposite that receiving the cover.

10. The device as claimed in the preceding claim 9, wherein the receptacle contains disinfectant.

11. The device as claimed in claim 1, wherein a ruler is mounted on the receptacle or the support and comprises a gradation.

12. The device as claimed in claim 1, wherein the cover comprises several slots which can be arranged to form sectors or groups making it possible to classify or sort several instruments according to set criteria.

13. The device as claimed in claim 1, wherein the cover is flexible and elastic.

14. The device as claimed in claim 13, wherein the cover has a tab making it possible to easily mount and remove said cover on/from the receptacle by deforming said cover.

15. The device as claimed in claim 1, wherein the support, the receptacle, the ruler and/or the extension are made from a single piece to form a main body.

16. The device of claim 10, wherein the disinfectant is a disinfectant solution or a foam imbibed with a disinfectant solution.

17. The device of claim 2, wherein an extension fixedly attached to the ring is arranged to come to bear against the finger adjacent to the finger passing through the ring when the device is in place so as to prevent the ring and the device from turning in the in-use position of the device.

18. The device of claim 2, wherein the cover is formed of several pieces.

19. The device of claim 3, wherein the cover is formed of several pieces.

20. The device of claim 4, wherein the cover is formed of several pieces.
